(19) United States
(12) Patent Application Publication
(10) Pub. No.: US 2024/0377327 A1
(43) Pub. Date: Nov. 14, 2024

(54) SINGLE-CELL MULTI-OMICS TOOL WITH PHENOTYPIC ASSESSMENT

(71) Applicant: Yaw Ofosu Nyansa Ansong-Ansongton, Albany, CA (US)

(72) Inventor: Yaw Ofosu Nyansa Ansong-Ansongton, Albany, CA (US)

(21) Appl. No.: 18/600,677

(22) Filed: Mar. 9, 2024

(51) Int. Cl. G01N 21/64; C12N 5/00; C12Q 1/6834; C12Q 1/6844; C12Q 1/6869; G01N 15/10; G01N 33/53; G01N 33/68; G16B 30/10

(52) U.S. Cl. CPC G01N 21/6458; C12N 5/0012; C12Q 1/6834; C12Q 1/6844; C12Q 1/6869; G01N 15/10; G01N 33/53; G01N 33/68; G16B 30/10; G01N 2015/1006

(57) ABSTRACT

The present invention discloses a method for high-throughput single-cell analysis, integrating innovative techniques such as proximity extension analysis (PEA), unique molecular identifier tagging, and combinatorial optical cell identification. The method combines transmission-based digital holographic interference microscopy (DHIM) or Quantitative Phase Imaging (QPI) microscopy with microfluidic techniques to enable comprehensive single-cell analysis. Single cells encapsulated in droplets are uniquely identified using fluorescent microbeads, allowing for precise optical cell identification. Following optical analysis, droplets undergo cell lysis and molecular tagging, with mRNA attaching to magnetic beads and proteins binding to detection probes. The method employs PEA for detecting and quantifying protein interactions, and rolling circle amplification (RCA) for sequencing mRNA and proteins. The resulting sequencing information is correlated with phenomic data using unique molecular identifiers, enabling a comprehensive understanding of single-cell biology.

### CROSS-REFERENCE TO RELATED APPLICATIONS

Not applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### FIELD OF INVENTION

The present invention relates to single-cell multi-omics and, more particularly, to a single-cell multi-omics tool with phenotypic assessment.

### BACKGROUND OF THE INVENTION

B and T lymphocytes recognize foreign and self-antigens through their antigen receptors, governing their development, survival, and activation. The T Cell Receptor (TCR) and B Cell Receptor (BCR) are assembled from variable (V), diversity (D), and joining (J) gene segments in a process known as V(D)J recombination. Random addition or removal of nucleotides at the complementarity determining region 3 (CDR3) largely determines antigen specificity. The significant diversity of the BCR and TCR repertoire, estimated at >10\hat{ }12, suggests that cells with the same receptor sequence are clonally related and constitute a clonotype. Thus, the receptor sequence serves as a unique clonal identifier or ‘clonal barcode,’ providing information on antigen specificity and clonal ancestry.

Sequencing BCR or TCR in parallel with their transcriptome offers insights into adaptive immune responses in diseases like infectious diseases, autoimmune disorders, and cancer. SmartSeq2 is commonly used to link paired antigen receptor sequences with gene expression profiles, but its throughput is limited to tens or hundreds of cells, and the cost per cell is relatively high.

Recent advancements in high-throughput single-cell RNA sequencing methods allow thousands of cells to be captured and sequenced at a fraction of the cost. However, short-read sequencing methods fail to sufficiently sequence the V(D) J regions of TCR and BCR transcripts, limiting the determination of clonotypic information from large numbers of lymphocytes. Long-read sequencing technologies offer potential solutions but suffer from higher error rates and lower sequencing depth.

Improved methodologies for high-throughput and multiplexed targeted long-read single-cell sequencing, particularly for cellular phenotyping, are needed.

Nucleic amplification-based research techniques, like Next Generation Sequencing, yield vast data on genomes and complicated samples, often from single cells. There's a need for accurate, scalable sequencing approaches, especially for simultaneous analysis of RNA, DNA, and proteins.

Current single-cell multi-omics tools lack complete phenotypic assessment, missing critical information for establishing genome-phenome relationships. By omitting single-cell phenomic data, these systems fail to understand which gene variants affect phenotype and miss out on key information for inferring health, disease, crop yields, and evolutionary fitness.

There is a need for a tool providing phenomic assessments of the morphological, physiological, and biochemical characteristics of single cells.

### SUMMARY

The method presented herein represents a significant advancement in single-cell analysis, offering several key innovations and advantages over existing techniques. Unlike conventional approaches, which often lack the ability to comprehensively assess single-cell biology, the present method integrates cutting-edge technologies to provide a holistic view of cellular function and behavior.

One of the primary innovations of the method is the integration of DHIM or QPI microscopy with microfluidic techniques, allowing for precise measurement of cellular deformability and biophysical properties at high throughput. This combination enables researchers to analyze cellular biomechanics in unprecedented detail, providing insights into disease mechanisms and cellular responses.

Furthermore, the method introduces a unique molecular identifier tagging approach, which facilitates the correlation of sequencing data with specific droplets and their phenomic information. This innovative tagging method enhances the accuracy and reliability of sequencing results, enabling researchers to confidently analyze gene expression patterns and protein profiles at the single-cell level.

Additionally, the method incorporates a combinatorial optical cell identification strategy, enabling the precise identification of individual droplets based on unique combinations of cell sizes, color combinations, and particle numbers. This approach enhances the efficiency and accuracy of single-cell analysis, facilitating the identification of rare cell populations and the detection of subtle cellular changes.

### BRIEF DESCRIPTION OF DRAWINGS

FIG. 1 is a schematic view of a multi-omics tool according to an embodiment of the present invention;

FIG. 2 is a schematic view of a method of tagging individual cells with a unique identification according to an embodiment of the present invention

FIG. 3 is a schematic view of process steps occurring on a microfluidics chip of the tool of FIG. 1;

FIG. 4 is another schematic view thereof.

### DETAILED DESCRIPTION OF THE INVENTION

Provided below is the non-limiting exemplary embodiment of the present disclosure and a reference will now be made in detail to specific embodiment or features, examples of which are illustrated in accompanying drawings. Wherever possible, corresponding or similar references numbers will be used throughout the drawings to refer to the same or corresponding parts. Moreover, reference of the various elements described herein, are made collectively or individually when there may be more than one element of the same type. However, such references are merely exemplary in nature. It may be noted that any reference to element in the singular may also be constructed to relate to the plural and vice-versa without limiting the scope of the disclosure to the exact number or type of such element unless set forth explicitly in the appended claim.

The following detailed description is of the best currently contemplated modes of carrying out exemplary embodiments of the invention. The description is not to be taken in a limiting sense but is made merely for the purpose of illustrating the general principles of the invention since the scope of the invention is best defined by the appended claims.

Single cells may each be encapsulated with a unique ID, such as a unique molecular identifier (UMI). The ID system may be based on molecular ID tags or on unique optics-based identifiers, such as optical beads/fluorescent beads/quantum dots. Beads or bead combinations of different colors, sizes, and shapes may be placed in each unique droplet to enable identification. These identifiers are incorporated into each droplet containing a single cell, ensuring that each cell has a unique identifying feature.

This information is converted into a single code representing the cell. The code serves as a representation of the cell's identity and can be used for tracking and analysis purposes. During, before, or after a biophysical measurement, each cell's ID is determined. The biophysical data corresponding to each cell's ID may be stored in a database. The database serves as a repository for all the measured biophysical properties of individual cells, facilitating further analysis and interpretation.

The cell's shape, flexibility, volume, deformability, and other phenomic features may be measured by optics, such as by quantitative phase imaging methods.

Cell phenomic data and biophysical measurements may alternatively (or also) be measured using microfluidics. The droplets may be directed either passively or actively into a microfluidics device to a measurement chamber in a microfluidic channel. Multi-parametric methods may utilize a multi-terminal measurement of cell behavior and/or the current across the microfluidic channel to quantify cell diameter, deformation measurements, and recovery measurements post-deformation. The measurement chamber may in some cases have a larger height and width than the channels. The measurement chamber may have a one-way valve.

The measurement chamber may also measure cell density using magnetic levitation, with cell levitation height in the chamber based on density.

The cells may be directed into a microfluidic channel, where they may be further encapsulated in a larger droplet (in any combination) with a magnetic bead, lysis agent, protein antibodies, and/or molecular tags for binding to DNA. The cell may be lysed by the lysis agent and mRNA may hybridize to the magnetic bead having an attached poly-T oligo. The proteins also may hybridize to antibodies having proximity extension probes and DNA may bind to specific molecular probes. Epigenomic measurements may also be paired with the phenomic measurement. The droplet may be channeled into and hydrodynamically trapped in a chamber in the chip. Each trap has a molecular ID in a specific location. The cell's unique molecular or optical ID may be measured and correlated with its unique location in the chip. After this, the droplet may be broken, e.g., with a demulsifier. Each hybridization chamber has two sub-chambers, one with a bead that magnetically attracts the mRNA; the other hybridizes with the proximity extension protein probes. The mRNA and proteins may then be pooled into two separate tubes respectively.

The molecular information associated with each droplet's ID within hydrodynamic traps is correlated with phenomic measurements. The acquisition of genomic and transcriptomic data from single cells is outlined, alongside various methods employed for molecular analysis.

Genomic and transcriptomic information is typically obtained through sequencing techniques, revealing the genetic makeup and gene expression profiles of individual cells. DNA sequencing elucidates the cell's genetic sequence, uncovering mutations, variations, and other genomic features. Transcriptomic sequencing captures RNA molecules present in the cell, reflecting actively expressed genes.

Concurrently, phenomic data, encompassing observable cellular characteristics, is measured, including but not limited to cell shape, size, and deformability. The correlation between molecular data and phenomic measurements facilitates the linkage of genetic and molecular features with observable cell traits.

An array of molecular measurement tools, such as but not limited to qPCR and imaging-based techniques, are utilized to obtain genomic and transcriptomic data. qPCR quantifies specific DNA or RNA sequences, offering insights into gene expression levels and genetic variations within single cells.

Furthermore, proteomic measurements, investigating the proteins expressed by cells, employ techniques such as but not limited to proximity extension assay, mass spectrometry, and western blotting. These methods identify and quantify proteins in individual cells, providing valuable information on cellular functions and signaling pathways.

The integration of molecular and phenomic data through advanced sequencing and proteomic techniques allows for a comprehensive understanding of cellular behavior, gene expression dynamics, and protein interactions at the single-cell level.

In another embodiment, bioinformatics and data analysis are conducted either on a local computer or in cloud-based environments to facilitate automated processing of data acquired from single-cell analysis. This analytical process involves the utilization of computational algorithms and software tools to extract meaningful insights from large datasets generated during experiments.

By employing bioinformatics techniques, genomic, transcriptomic, and proteomic data obtained from single cells can be efficiently analyzed. This analysis may include tasks such as sequence alignment, variant calling, gene expression quantification, and pathway analysis, among others. Additionally, data analysis tools enable the visualization of results through graphical representations, aiding in the interpretation and communication of findings.

Further, bioinformatics and data analysis are integrated with CRISPR gene editing libraries and other small molecule libraries to facilitate drug discovery efforts. By correlating genomic and proteomic data with phenotypic information obtained from single-cell analyses, researchers can identify potential drug targets and pathways for therapeutic intervention. This integration of bioinformatics with experimental data enhances the efficiency and effectiveness of drug discovery processes, potentially leading to the development of novel treatments for various diseases.

In some embodiments, mechatronics plays a pivotal role in automating various aspects of the experimental process. One crucial function of mechatronics involves the automation of microfluidic chip insertion into the system. This automation ensures precise and consistent placement of the microfluidic chips, optimizing experimental conditions and reducing human error. Mechatronics is also responsible for controlling the pressures within the microfluidic chambers. By adjusting these pressures, mechatronics ensures the proper encapsulation of single cells within droplets and regulates the flow of fluids through the microfluidic channels. This precise control is essential for maintaining optimal conditions for single-cell analysis and minimizing experimental variability. Furthermore, mechatronics facilitates the pooling of data and results obtained from the single-cell analysis. By integrating data acquisition systems and computational algorithms, mechatronics automates the collection, processing, and storage of experimental data. This automation streamlines the data analysis workflow, enabling researchers to efficiently extract valuable insights from the experimental results.

The microfluidics chip may be prototyped by patterning the chip design on a silicon wafer using photolithography as well as transferring the pattern onto a PDMS mold via soft lithography. Entry and exit points may be punched in the PDMS. The measurement chamber may contain two magnetic levitation magnets with or without a non-ionic paramagnetic medium. The mRNA/DNA/Proteome trapping chambers may be treated with relevant molecular biology reagents after plasma treatment. The PDMS device may be attached to glass. Mass-producing the chip may require fabrication of mold or master. This may be performed with mechanical (micro-cutting; ultrasonic machining), energy-assisted methods (electro-discharge machining, micro-electrochemical machining, laser ablation, electron beam machining, focused ion beam (FIB) machining), traditional micro-electromechanical systems (MEMS) processes, as well as mold fabrication approaches for curved surfaces. Production may be low-volume (casting, lamination, laser ablation, 3D printing) or high-volume (hot embossing, injection molding, and film or sheet operations). A mechanical system may hold the optical setup in place. The optical setup may include lasers and lenses and the system may be compact.

The complete hardware system may comprise a printed circuit board (PCB) with relevant software installed. Software may be configured for a cloud software backend, along with a database and/or local computer software. An API may enable communication between the device and the analytics package and a front-end may enable display and communication between the user and the backend.

To use the system according to an embodiment of the present invention, a person may obtain cells. The cells may be placed into a special ‘cell holder’ made of plastic or any other material. The cell holder may be inserted into the device. At this point the microfluidic chip may be inserted into the device. Optical beads/droplet oil and continuous phase material such as water in a pre-made chamber may also be inserted into the device.

In some embodiments, after a ‘start’ button is pressed, the single cells in the cell holder are optically assessed for size and the automated system pushes the cells across the microfluidic channel. The cells may be droplet encapsulated and may flow into the phenomic chamber where various optical and biophysical measurements on the cell feature may be performed. This may alternatively be done using a microfluidic channel in a high throughput manner on multiple cells. The cells may be molecularly tagged with a transcriptome and/or genome and/or chromatin information and/or proteome is separated and pooled into different channels. The liquid output in different tubes may be sequenced and/or analyzed for results. The uploaded results may be analyzed with data analysis software and conclusions may be drawn.

In some embodiments, molecular measurements (e.g., protein measurements or transcriptome measurements) may be done alone, without measuring cell phenomic features. Alternatively, the molecular measurements (mRNA/Protein measurements) may be performed first before performing the phenomic measurements; and the cell ID may be measured before or after the phenomic measurement.

In some embodiments, the phenomic tool may be used alone, with the cells being measured just for phenomic features, without sequencing.

In some embodiments, optics may be used alone to measure just phenomic features. In some embodiments, the optical component may be used first to measure cell size before encapsulation and much more detailed phenomic and molecular measurements. In alternative embodiments, optics may be used first, followed by tissue culture, then optics and/or microfluidics. Sequencing, bioinformatics, and data analysis, and mechatronics may then be performed in sequence.

In some embodiments, tissue culture is followed by optics and/or microfluidics. Sequencing, bioinformatics and data analysis, and mechatronics may then be performed in sequence.

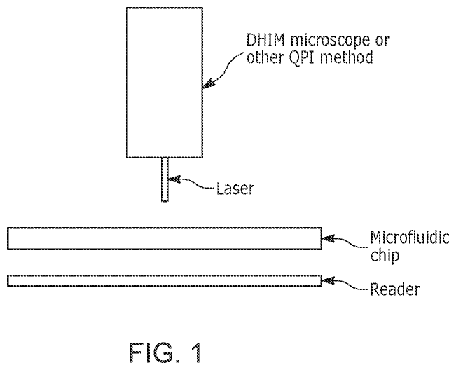

The method and system disclosed herein may further comprise printing the cells on a board with oligos that depict x, y and z positions. In some embodiments the cells may be printed to a board first, and the characterization using optics may then be performed. In other embodiments, the cell features may be biophysically characterized via microfluidics and optics and then printed to a board. Referring now to FIGS. 1 through 4, FIG. 1 illustrates a tool according to an embodiment of the present invention, for measuring biophysical features, comprising a transmission-based digital holographic interference microscope (DHIM) microscope 10 or other Quantitative Phase Image (QPI) microscopy device having a measurement chamber on a microfluidic chip 14. The microscope 10 directs a laser beam 12 through the microfluidic chip 14 onto a reader 16, measuring deformability. The DHIM microscope 10 may be high throughput, capturing several frames per second, and may have fluorescence measurement capabilities.

In the method described for high-throughput single-cell analysis, it is emphasized that biophysical measurements are not solely limited to techniques such as transmission-based digital holographic interference microscopy (DHIM) or Quantitative Phase Imaging (QPI). Instead, biophysical features can also be assessed using microfluidic techniques. Microfluidics may provide a versatile platform for manipulating and analyzing small volumes of fluids, including individual cells, within microscale channels and chambers. In the context of single-cell analysis, microfluidic devices may offer the capability to perform diverse biophysical measurements on individual cells as they traverse through the channels.

One common application of microfluidic-based single-cell analysis involves leveraging hydrodynamic principles to evaluate biophysical properties such as cell deformability, size, and mechanical characteristics. This is achieved by subjecting cells to controlled flow rates or pressures within narrow channels, enabling the assessment of cell stiffness or deformability. Additionally, microfluidic platforms can integrate optical or electrical sensors to measure various biophysical parameters. For instance, optical sensors within microfluidic channels can monitor changes in cell morphology or refractive index, providing insights into cell shape, volume, and optical density. Alternatively, electrical impedance-based sensors can assess cell size, shape, and membrane properties based on alterations in electrical impedance as cells traverse microfluidic channels. Furthermore, microfluidic devices offer the capability for multiparametric measurements, enabling concurrent evaluation of multiple biophysical features within a single platform. By incorporating diverse sensing modalities and fluidic manipulation techniques, microfluidic-based single-cell analysis systems provide researchers with versatile, scalable, and high-throughput tools for investigating cellular heterogeneity and function.

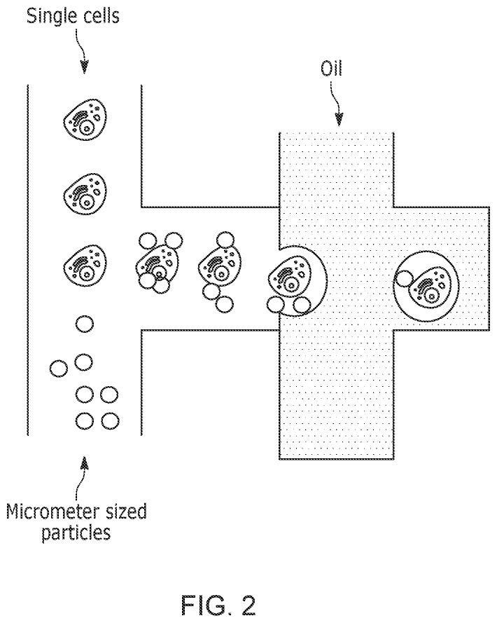

In an example shown in FIG. 2, cancer cells are combined with micrometer size particles and encapsulated in oil. Optical cell identification may be performed with fluorescent microbeads of different sizes, shapes, and colors encapsulated in multiple combinations in each droplet. By measuring the unique combinations of cell sizes, the color combinations, and the number of particles, each droplet can be identified.

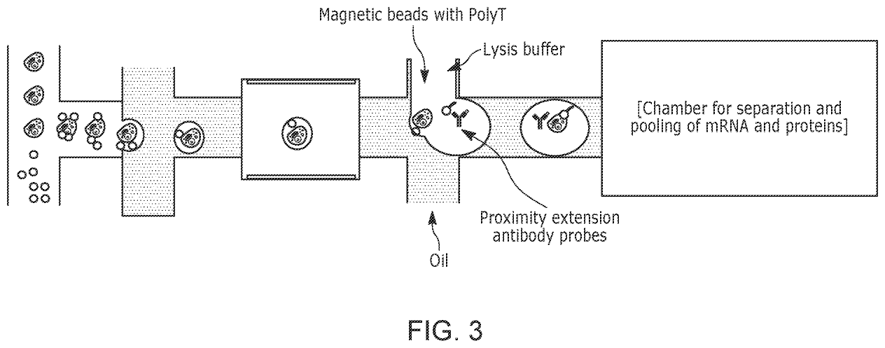

After encapsulation with their fluorescent ID as seen in FIG. 2, droplets enter a viewing chamber shown in FIG. 3, where they are optically analyzed. They are then encapsulated in a larger droplet where the cells are lysed in a lysis buffer; mRNA attaches to magnetic beads with Poly-T oligo and proteins bind protein detection probes and proximity extension antibody probes. The lysed cell contents with bound probes then enter a Chamber for separation and pooling of mRNA and protein.

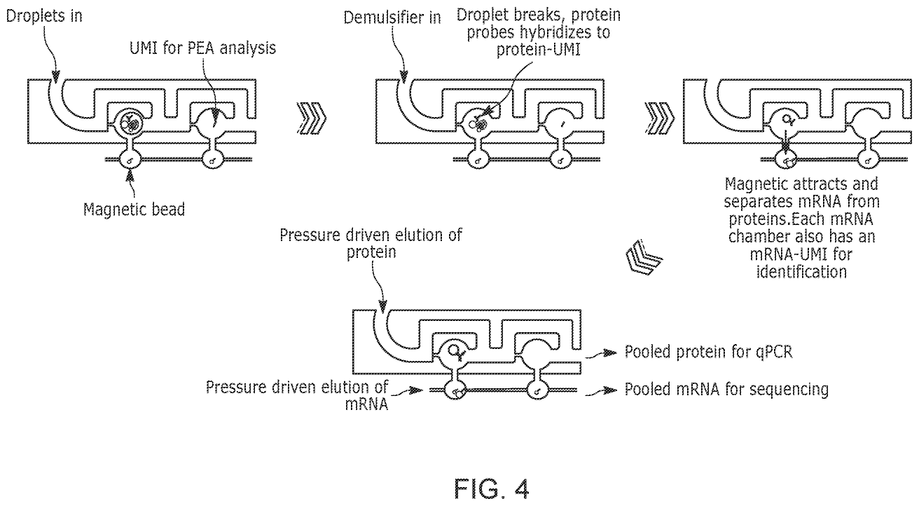

FIG. 4 illustrates a method of separating and pooling mRNA and protein. Droplets enter a chamber where they are imaged with their ID for the last time. Each hydrodynamic trap in the chamber has a unique molecular ID attached to it with proximity extension analysis (PEA). The droplet is destroyed, e.g., with a demulsifier, and mRNA and proteins separate magnetically. Each mRNA in each chamber binds to a molecular ID, along with each protein. The samples are pooled and sequenced and the sequencing information of each mRNA and protein is demultiplexed based on the location since each location in the well has a unique sequence. The sequence is also tied back to the optical ID in order to tie back to the phenomic information.

Furthermore, the sequencing information obtained includes mRNA and protein sequences, providing insights into gene expression profiles and protein compositions within individual cells. Additionally, genomic DNA sequencing may also be performed to analyze genetic mutations or variations. This sequencing data is demultiplexed based on the unique molecular identifiers attached to the hydrodynamic traps within the chamber, facilitating correlation with phenomic data.

PEA is used for detecting and quantifying the interaction between proteins or other molecules and is based on the principle of bringing two binding partners in close proximity and detecting the interaction by measuring the resulting signal.

PEA uses a pair of oligonucleotides that are conjugated to the two interacting molecules. These oligonucleotides have complementary sequences that can hybridize to form a double-stranded DNA molecule. When the two molecules interact, they bring the two oligonucleotides in close proximity, allowing them to hybridize and form a circular DNA molecule.

The circular DNA molecule is then amplified by rolling circle amplification (RCA) using a DNA polymerase enzyme. During RCA, the polymerase extends one of the primers around the circle, producing a long single-stranded DNA molecule that contains many copies of the complementary sequence. The resulting DNA molecule is then detected using various methods such as fluorescence or chemiluminescence.

PEA has several advantages over other methods for detecting protein-protein interactions. It is highly specific, as it relies on the binding of two specific molecules to each other. It is also sensitive, as it can detect interactions at low concentrations. PEA can be used for high-throughput screening of protein interactions, making it useful in drug discovery and other research applications.

The inventive tool may have a variety of uses. It may be used to produce single-cell molecular products such as transcripts, proteomes, and genomes. A scientist may use this tool to measure features of cancer cells and determine optimal patient outcomes based on cell features, and thus predict the appropriate personalized treatment option for the patient. This may involve all forms of human biological products. A pharmaceutical company may use the outcomes of treatment with their drugs to determine optimal drug targets to pursue. A water treatment facility may use this system to determine water quality. An agricultural company may use it to assess agricultural health and yields. A biologics manufacturer may use it to produce biological products such as proteins.

The foregoing description and accompanying figures illustrate the principles, embodiments and modes of operation of the invention. However, the invention should not be construed as being limited to the particular embodiments discussed above. Additional variations of the embodiments discussed above will be appreciated by those skilled in the art.

Therefore, the above-described embodiments should be regarded as illustrative rather than restrictive. Accordingly, it should be appreciated that variations to those embodiments can be made by those skilled in the art without departing from the scope of the invention as defined by the following claims.

The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of”.

The phrase “consisting essentially of” means that the composition or method may include additional ingredients and/or steps, but only if the additional ingredients and/or steps do not materially alter the basic and novel characteristics of the composition or method.

As used herein, the singular form “a”, “an” and “the” may include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the disclosure.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

1. A method for high-throughput single-cell analysis, comprising:
a. directing a laser beam through a microfluidic chip onto a reader within a transmission-based microscopy device, said microscopy device configured to measure cellular deformability and equipped with fluorescence measurement capabilities;
b. combining single cells with micrometer-sized particles and encapsulating them in oil within droplets, wherein each droplet is uniquely identified using fluorescent microbeads of varying sizes, shapes, and colors encapsulated in diverse combinations;
c. subjecting the encapsulated droplets to optical analysis within a viewing chamber, followed by encapsulation in larger droplets for cell lysis and subsequent attachment of mRNA to magnetic beads with Poly-T oligo, and binding of proteins to protein detection probes and proximity extension antibody probes;
d. separating and pooling mRNA and proteins within a chamber equipped with hydrodynamic traps, each trap possessing a unique molecular identifier attached through proximity extension analysis (PEA), followed by destruction of the droplets to separate mRNA and proteins magnetically, and pooling of samples for sequencing.

2. The method of claim 1, wherein, said microscopic device include digital holographic interference microscope (DHIM) or a Quantitative Phase Imaging (QPI)

3. The method of claim 1, wherein, PEA is utilized for detecting and quantifying protein interactions based on bringing two binding partners into close proximity and measuring resulting signals.

4. The method of claim 1, wherein PEA is employed to measure the interaction between proteins or other molecules by bringing the binding partners into close proximity, thereby facilitating the detection and quantification of specific molecular interactions.

5. The method of claim 1, wherein the pair of oligonucleotides used in proximity extension analysis (PEA) are conjugated to the interacting molecules, and upon interaction, hybridize to form a circular DNA molecule suitable for amplification.

6. The method of claim 1, wherein rolling circle amplification (RCA) is utilized to amplify the circular DNA molecule formed during proximity extension analysis (PEA), resulting in the generation of a long single-stranded DNA molecule containing multiple copies of the complementary sequence.

7. The method of claim 1, wherein the detection of the resulting DNA molecule, following rolling circle amplification (RCA), is performed using fluorescence-based methods to visualize and quantify the amplified DNA strands.

8. The method of claim 1, wherein the unique molecular identifier tagging approach comprises attaching distinct molecular identifiers to each hydrodynamic trap within the chamber, facilitating the correlation of sequencing data with specific droplets and their respective phenomic information.

9. The method of claim 1, wherein the combinatorial optical cell identification method involves encapsulating single cells with micrometer-sized particles and fluorescent microbeads of varying sizes, shapes, and colors in multiple combinations within droplets, enabling the identification of individual droplets based on unique combinations of cell sizes, color combinations, and the number of particles.

10. The method of claim 1, wherein the microfluidic techniques for biophysical measurements include assessing cell deformability, size, and mechanical properties using hydrodynamic principles within microfluidic channels, thereby providing insights into cellular biomechanics at the single-cell level.

11. The method of claim 1, further comprising performing quality control measures on the amplified DNA molecules to ensure the accuracy and reliability of sequencing results, wherein quality control measures include sequence alignment, error correction, and removal of background noise.

12. The method of claim 1, wherein the fluorescence or chemiluminescence-based detection of the resulting DNA molecule, following rolling circle amplification (RCA), is conducted using a high-throughput imaging system capable of analyzing multiple samples simultaneously, thereby increasing the efficiency of data acquisition and analysis.
